(19) United States
(12) Patent Application Publication
(10) Pub. No.: US 2023/0064583 A1
(43) Pub. Date: Mar. 2, 2023

(54) AIR PURIFICATION APPARATUS

(71) Applicant: Shang Honng Technology Co., Ltd., Hsinchu County (TW)

(72) Inventor: Lun Tsai, Hsinchu County (TW)

(21) Appl. No.: 17/407,205

(22) Filed: Aug. 20, 2021

(51) Int. Cl. A61L 9/20; B01D 53/00; B01D 53/86

(52) U.S. Cl. CPC A61L 9/205; B01D 53/007; B01D 53/86; B01D 2255/802; A61L 2209/12; A61L 2209/21; A61L 2209/15

(57) ABSTRACT

This disclosure discloses an air purification apparatus, and the air purification apparatus includes an inner housing, a plurality of photocatalytic reactors and a light source. The inner housing is porous to allow air flow to pass. The photocatalytic reactors are filled in the inner housing. The photocatalytic reactors respectively have a photocatalytic layer formed thereon. The light source is disposed in the inner housing and surrounded by the photocatalytic reactors. The light source is configured to irradiate photocatalytic reactors to activate the photocatalytic layers on the photocatalytic reactors.

### BACKGROUND

### 1 Technical Field

This disclosure generally relates to an air purification apparatus, more particularly, to an air purification apparatus capable of effectively increasing the chance of airflow colliding with photocatalyst and improving purification effect.

### 2. Description of the Related Art

Ultraviolet rays have a good sterilization and disinfection effect. Ultraviolet disinfection lamps have been widely used at present and are the best method for disinfection and sterilization of infectious viruses. Photocatalyst is a catalyst that uses light energy to carry out a catalytic reaction. The photocatalyst is first coated or sprayed on the surfaces of objects to form thin films, which can activate to reduce the foreign substances attached to the surfaces of the objects through light energy to achieve the purpose of decontamination, sterilization, bacteriostasis or cleaning the surfaces of the objects.

### SUMMARY

Accordingly, the present disclosure provides an air purification apparatus capable of effectively increasing the chance of airflow colliding with photocatalyst and improving purification effect.

In one embodiment, the air purification apparatus of the present disclosure includes an inner housing, a plurality of photocatalytic reactors and a light source. The inner housing is porous to allow air flow to pass. The photocatalytic reactors are filled in the inner housing. The photocatalytic reactors respectively have a photocatalytic layer formed thereon. The light source is disposed in the inner housing and surrounded by the photocatalytic reactors. The light source is configured to irradiate photocatalytic reactors to activate the photocatalytic layers on the photocatalytic reactors.

In another embodiment, the air purification apparatus of the present disclosure includes two reactor sets and a plurality of light sources. The two reactor sets respectively have a casing and a plurality of photocatalytic reactors filled in the casing. The photocatalytic reactors respectively have a photocatalytic layer formed thereon. The casings are porous to allow air flow to pass. The light sources are sandwiched between the two casings. The light sources are configured to irradiate photocatalytic reactors to activate the photocatalytic layers on the photocatalytic reactors.

In further another embodiment, the air purification apparatus of the present disclosure includes a plurality of photocatalytic reactors and at least one light source. The photocatalytic reactors are spaced from each other. The photocatalytic reactors respectively include a corrugated plate with a photocatalytic layer formed thereon. The at least one light source is configured to irradiate photocatalytic reactors to activate the photocatalytic layers on the photocatalytic reactors.

### BRIEF DESCRIPTION OF THE DRAWINGS

Other objects, advantages, and novel features of the present disclosure will become more apparent from the following detailed description when taken in conjunction with the accompanying drawings.

FIG. 1a is an exploded view of an air purification apparatus according to a first embodiment of the present disclosure.

FIG. 1b is an elevated perspective view of the air purification apparatus according to the first embodiment of the present disclosure.

FIG. 1c is an exploded view of the air purification apparatus according to another aspect of the first embodiment of the present disclosure.

FIG. 2 is a schematic view illustrating that air is inhaled into and exhaled from the air purification apparatus following the direction indicated by the arrows according to the first embodiment of the present disclosure.

FIG. 3 is an exploded view of an air purification apparatus according to a second embodiment of the present disclosure.

FIG. 4 is an elevated perspective view of the air purification apparatus according to the second embodiment of the present disclosure.

FIG. 5 is a schematic view illustrating that air is inhaled into and exhaled from the air purification apparatus following the direction indicated by the arrows according to the second embodiment of the present disclosure.

FIG. 6 is an exploded view of the air purification apparatus according to another aspect of the second embodiment of the present disclosure.

FIG. 7 is an elevated perspective view of the air purification apparatus according to another aspect of the second embodiment of the present disclosure.

FIG. 8 is an exploded view of an air purification apparatus according to a third embodiment of the present disclosure.

FIG. 9 is an elevated perspective view of the air purification apparatus according to the third embodiment of the present disclosure.

FIG. 10 is a schematic view illustrating that air is inhaled into and exhaled from the air purification apparatus following the direction indicated by the arrows according to the third embodiment of the present disclosure.

FIG. 11 is a schematic view of the air purification apparatus according to another aspect of the third embodiment of the present disclosure, wherein air is inhaled into and exhaled from the air purification apparatus following the direction indicated by the arrows.

FIG. 12 is a flow chart of a method of manufacturing a photocatalytic reactor according to some embodiments of the present disclosure.

FIG. 13 is a flow chart of a method of manufacturing a mini cold cathode tube according to some embodiments of the present disclosure.

### DETAILED DESCRIPTION OF THE PREFERRED EMBODIMENT

An air purification apparatus, a method of manufacturing a photocatalytic reactor and a method of manufacturing a mini cold cathode tube according to embodiments of the present disclosure are described below. However, the embodiments provided in the present disclosure are merely illustrative of examples of the air purification apparatus, the method of manufacturing a photocatalytic reactor and the method of manufacturing a mini cold cathode tube of the present disclosure. The embodiments provided in the present disclosure are not intended to limit the scope of the present disclosure.

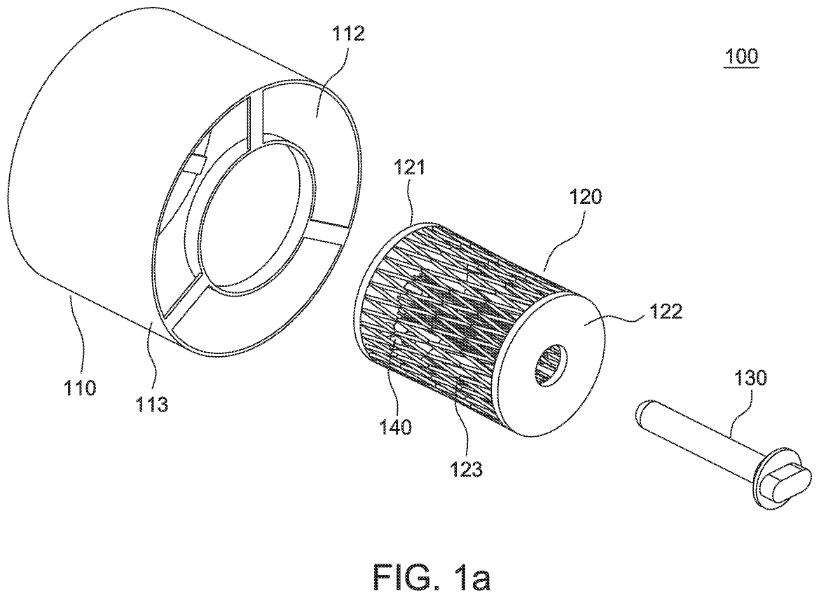

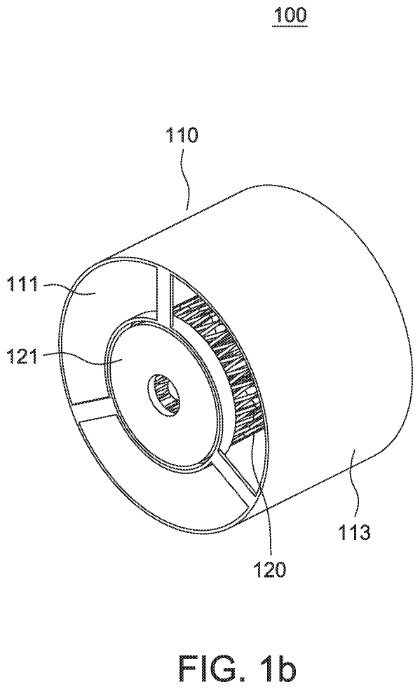

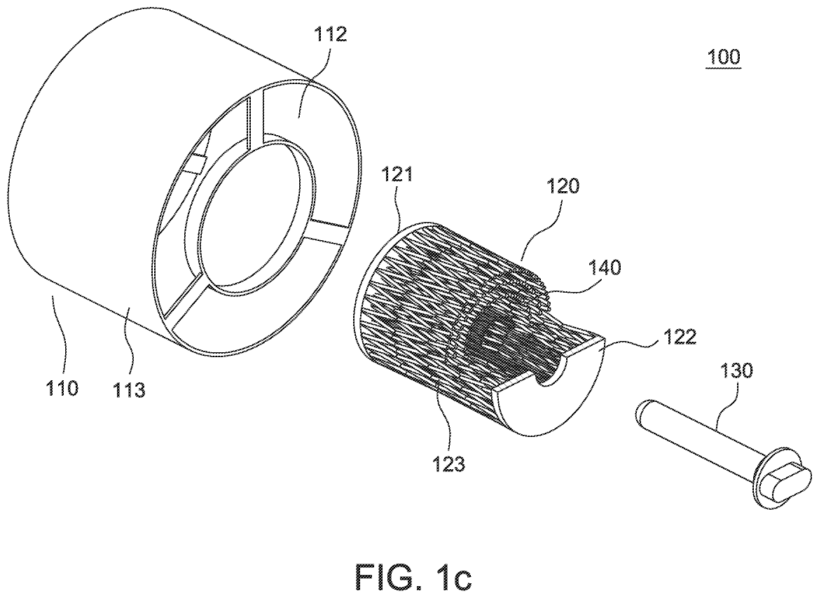

FIG. 1a is an exploded view of an air purification apparatus according to a first embodiment of the present disclosure. FIG. 1b is an elevated perspective view of the air purification apparatus according to the first embodiment of the present disclosure. The air purification apparatus 100 includes a main body 110, an inner housing 120, a light source 130 and a plurality of photocatalytic reactors 140.

The main body 110 is hollow and of cylindrical shape. The main body 110 has an annular side face 113 and two opposing first and second ends 111, 112 coupled to the side face 113. The side face 113 has an inner surface that is a reflective surface for reflecting ultraviolet light. Further, the side face 113 is arranged to stop air flow through. The first end 111 is an open end and configured to function as an air inlet. The second end 112 is an open end and configured to function as an air outlet. This means that air may enter the main body 110 from the first end 111 and be confined in the main body 110 by the side face 113. The air finally leaves the main body 110 from the second end 112.

The inner housing 120 is of cylindrical shape and disposed in the main body 110. The inner housing 120 is hollow and has an annular side face 123 and two opposing first and second ends 121, 122 coupled to the side face 123. The annular side face 123 faces the annular side face 113 of the main body 110 and the first and second ends 121, 122 respectively face the first and second ends 111, 112 of the main body 110. The side face 123 is porous to allow air flow to pass.

The photocatalytic reactors 140 are ball-shaped and filled in the inner housing 120. The photocatalytic reactors 140 respectively have a photocatalytic layer formed thereon. The photocatalytic layers may be formed on the photocatalytic reactors 140 by coating or spraying process. The photocatalytic layers on the photocatalytic reactors 140 may activate to reduce the foreign substances in the air through light energy.

The light source 130 is disposed in the inner housing 120 and surrounded by the photocatalytic reactors 140. In one embodiment, the light source 130 may be an ultraviolet lamp. The light source 130 irradiates the photocatalytic reactors 140 so that the photocatalytic layers on the photocatalytic reactors 140 are activated by ultraviolet light to decontaminate or clean the air flowing pass the photocatalytic reactors 140.

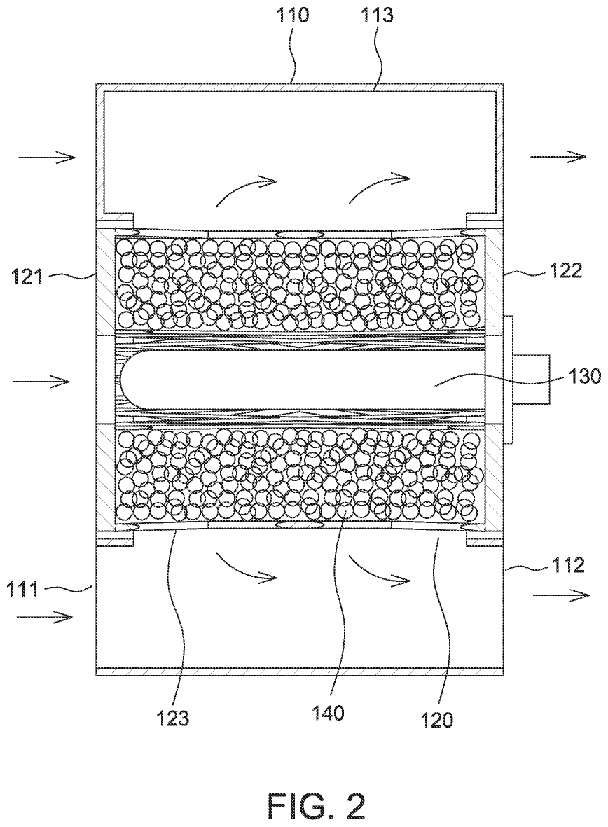

Now please refer to FIG. 2. In the air purification apparatus 100 of the present disclosure, air enters the air purification apparatus 100 from the first end 111 of the main body 110. The air may be inhaled into the main body 110 by a fan equipped in the main body 110 (not shown in the figure). The direction of arrows shown in the figure indicates that of the air flow. The air inhaled may enter the inner housing 120 through the porous side face 123. The light source 130 irradiates the photocatalytic reactors 140 to activate the photocatalytic layers thereon. The air entering the inner housing 120 may contact and react with the activated photocatalytic layers on the photocatalytic reactors 140. Accordingly, the air inhaled is processed to be purified by the photocatalytic reactors 140. The purified air will finally leave the air purification apparatus 100 from the second end 112 of the main body 110.

In the air purification apparatus 100 of the present disclosure, the light source 130 may be a hot cathode tube lamp or a cold cathode tube lamp. In other embodiments, the light source 130 may be a light bar composed of a plurality of light-emitting diodes (LEDs). In other words, the light source 130 may be selected from the group consisting of a hot cathode tube lamp, a cold cathode tube lamp, a light bar composed of LEDs and combinations thereof. The light source 130 may be an ultraviolet lamp to generate ultraviolet light with a wavelength smaller than 450 nm to better activate the photocatalytic layers on the photocatalytic reactors 140.

In the air purification apparatus 100 of the present disclosure, the inner surface of the side face 113 of the main body 110 may be a reflective surface to reflect the ultraviolet light. This may utilize the ultraviolet light emitting from the light source 130 more efficiently.

In the air purification apparatus 100 of the present disclosure, the bodies of the photocatalytic reactors 140 may be made of silicon dioxide or metal oxide through which light may penetrate or reflect. The metal oxide making up the bodies of the photocatalytic reactors 140 may be aluminum oxide, zirconium oxide, magnesium oxide or calcium oxide and combinations thereof. The photocatalytic layers on the photocatalytic reactors 140 may be made of titanium dioxide, zinc oxide or tungsten oxide and combinations thereof.

In the air purification apparatus 100 of the present disclosure, the photocatalytic reactors 140 may be spherical and unevenly distributed in the inner housing 120 as shown in the FIG. 2. In other embodiments, the photocatalytic reactors 140 may be cube-shaped, rod-shaped, fiber-shaped or sheet-shaped and stacked in the inner housing 120.

Please refer to FIG. 1c, which is an exploded view of the air purification apparatus according to another aspect of the first embodiment of the present disclosure. The photocatalytic reactors 140 may include more than three layers of mesh structure. The mesh structure has an aperture size of less than 10 mm and misalignment of apertures in the layers of mesh structure is greater than 0.1 mm. The mesh structures may be composed of aluminum, iron, titanium, nickel and alloys thereof. The mesh structures may also be made of ceramic. There are two layers of coating including a first layer of coating and a second layer of coating formed on the mesh structure. The first layer of coating is configured to increase surface areas and is composed of silicon dioxide (SiO₂), calcium oxide (CaO), zinc oxide (ZnO), magnesium oxide (MgO), aluminum oxide (Al₂O₃) and combinations thereof. The second layer of coating is formed on the first layer and composed of photocatalytic layer.

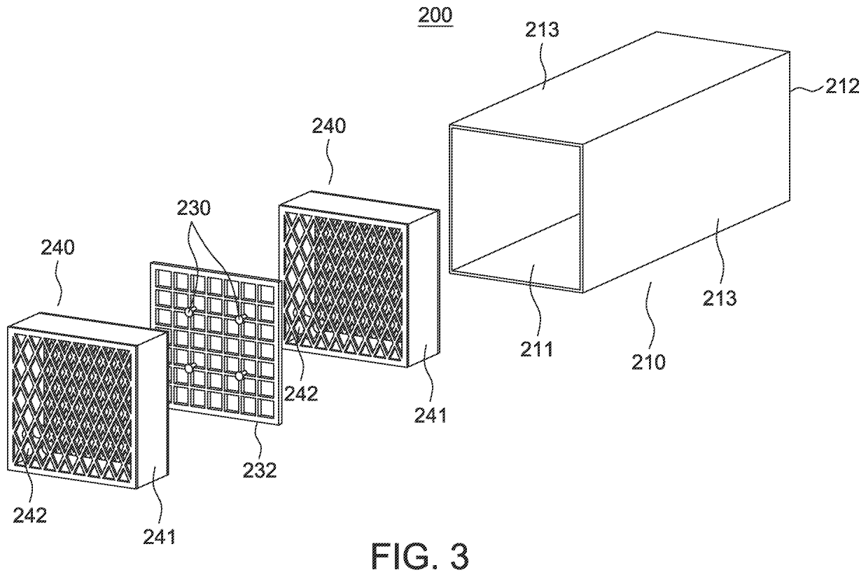

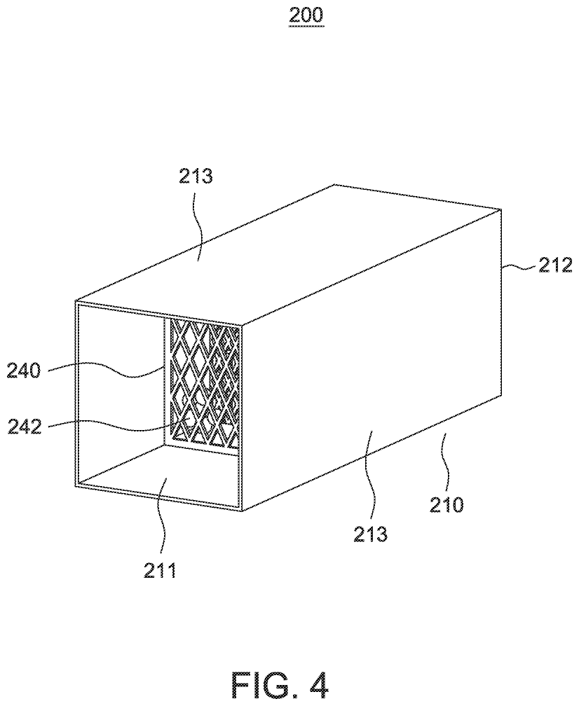

FIG. 3 is an exploded view of an air purification apparatus according to a second embodiment of the present disclosure. FIG. 4 is an elevated perspective view of the air purification apparatus according to the second embodiment of the present disclosure. The air purification apparatus 200 includes a main body 210, a plurality of light sources 230 and two reactor sets 240.

The main body 210 is hollow and of cuboidal shape. The main body 210 has a plurality of side faces 213 and two opposing first and second ends 211, 212 coupled to the side faces 213. The side faces 213 respectively have an inner surface that is a reflective surface for reflecting ultraviolet light. Further, the side faces 213 are arranged to stop air flow through. The first end 211 is an open end and configured to function as an air inlet. The second end 212 is an open end and configured to function as an air outlet. This means that air may enter the main body 210 from the first end 211 and be confined in the main body 210 by the side faces 213. The air finally leaves the main body 210 from the second end 212.

The reactor sets 240 are disposed in the main body 210 and respectively have a casing 241 and a plurality of ball-shaped photocatalytic reactors 242 filled in the casing 241. The casings 241 respectively have a shape of rectangular plate and may be transparent to ultraviolet light. The casings 241 face the first and second ends 211, 212 of the main body 210 respectively. The casings 241 are porous to allow air flow to pass.

The photocatalytic reactors 242 respectively have a photocatalytic layer formed thereon. The photocatalytic layers may be formed on the photocatalytic reactors 242 by coating or spraying process. The photocatalytic layers on the photocatalytic reactors 242 may activate to reduce the foreign substances in the air through light energy.

The light sources 230 are disposed in the main body 210 and sandwiched between the casings 241 of the reactor sets 240. The light sources 230 irradiates the photocatalytic reactors 242 in the reactor sets 240 so that the photocatalytic layers on the photocatalytic reactors 242 are activated by ultraviolet light to decontaminate or clean the air flowing pass the photocatalytic reactors 242.

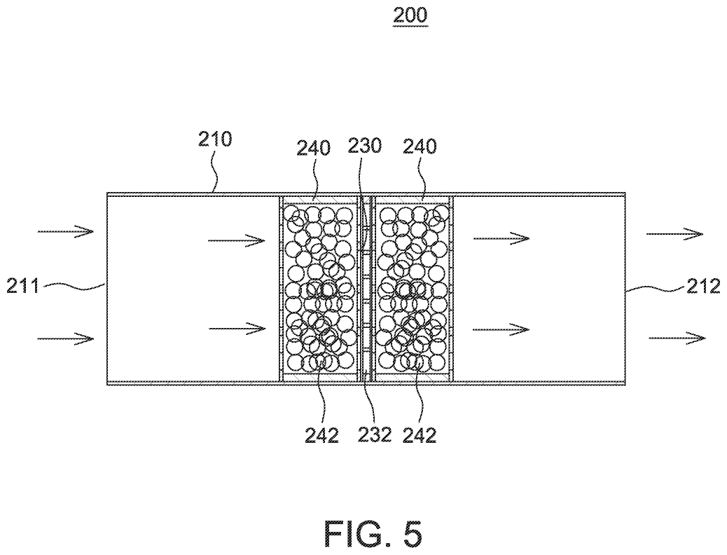

Now please refer to FIG. 5. In the air purification apparatus 200 of the present disclosure, air enters the air purification apparatus 200 from the first end 211 of the main body 210. The air may be inhaled into the main body 210 by a fan equipped in the main body 210 (not shown in the figure). The direction of arrows shown in the figure indicates that of the air flow. The air inhaled may enter the casings 241 to contact the photocatalytic reactors 242 therein. The inhaled air passes the casing 241 near the first end 211, the light sources 230 and the casing 241 near the second end 212 of the main body 110 in sequence. The light sources 230 irradiate the photocatalytic reactors 242 to activate the photocatalytic layers thereon. The air entering the casings 241 contacts and reacts with the activated photocatalytic layers on the photocatalytic reactors 242. Accordingly, the air inhaled is processed to be purified by the photocatalytic reactors 242. The purified air will finally leave the air purification apparatus 200 from the second end 212 of the main body 210.

In the air purification apparatus 200 of the present disclosure, the light sources 230 may be ultraviolet LEDs. In other embodiment, the light sources 230 may be hot cathode tube lamps or cold cathode tube lamps. In other words, the light sources 230 may be selected from the group consisting of hot cathode tube lamps, cold cathode tube lamps, LEDs and combinations thereof. The light sources 230 are provided to generate ultraviolet light with a wavelength smaller than 500 nm to better activate the photocatalytic layers on the photocatalytic reactors 242. In addition, the light sources 230 may be ones with the same or different wavebands so as to achieve better combination of light intensity and driving energy. The light sources 230 may be attached to a frame 232 so as to arrange in a checkerboard shape. The frame 232 is designed based on the principle of the smallest cross-sectional area, and the total shielding area shielding by the frame 232 is less than 50% of the total ventilation area of the frame 232. In one embodiment, the light sources 230 are ones having more than two different wavebands that are alternately arranged in the checkerboard shape.

In the air purification apparatus 200 of the present disclosure, the inner surfaces of the side faces 213 of the main body 210 may be reflective surfaces to reflect the ultraviolet light. This may utilize the ultraviolet light emitting from the light sources 230 more efficiently.

In the air purification apparatus 200 of the present disclosure, the bodies of the photocatalytic reactors 242 may be made of silicon dioxide or metal oxide through which light may penetrate or reflect. The metal oxide making up the bodies of the photocatalytic reactors 242 may be aluminum oxide, zirconium oxide, magnesium oxide or calcium oxide and combinations thereof. The photocatalytic layers on the photocatalytic reactors 242 may be made of titanium dioxide, zinc oxide or tungsten oxide and combinations thereof.

In the air purification apparatus 200 of the present disclosure, the photocatalytic reactors 242 may be spherical and unevenly distributed in the casings 241. In other embodiments, the photocatalytic reactors 242 may be cube-shaped, rod-shaped, fiber-shaped or sheet-shaped and stacked in the casings 241.

In addition, the photocatalytic reactors 242 may include more than three layers of mesh structure. The mesh structure has an aperture size of less than 10 mm and misalignment of apertures in the layers of mesh structure is greater than 0.1 mm The mesh structures may be composed of aluminum, iron, titanium, nickel and alloys thereof. The mesh structures may also be made of ceramic. There are two layers of coating including a first layer of coating and a second layer of coating formed on the mesh structure. The first layer of coating is configured to increase surface areas and is composed of silicon dioxide (SiO₂), calcium oxide (CaO), zinc oxide (ZnO), magnesium oxide (MgO), aluminum oxide (Al₂O₃) and combinations thereof. The second layer of coating is formed on the first layer and composed of photocatalytic layer.

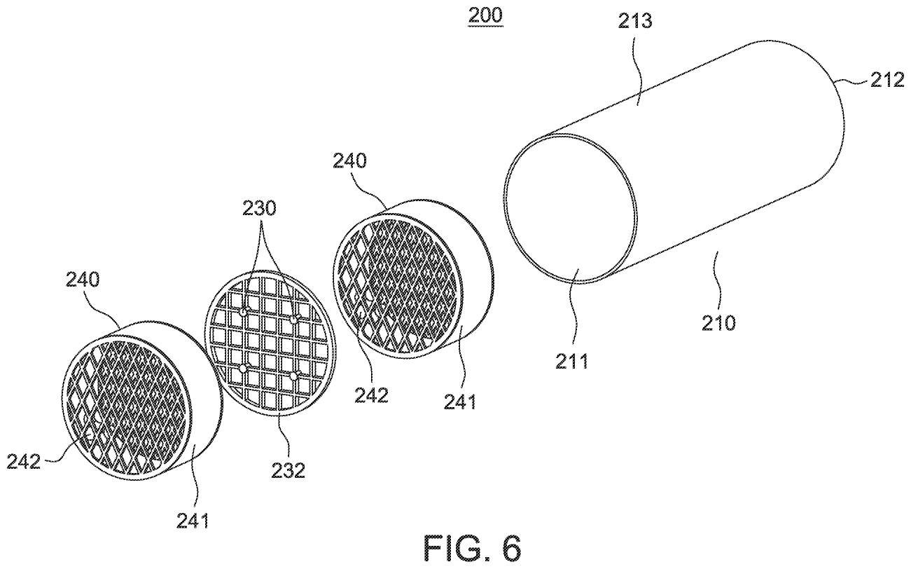

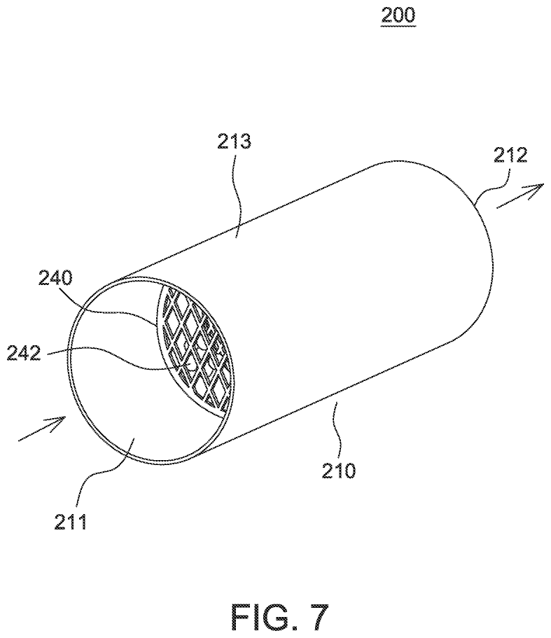

FIG. 6 is an exploded view of the air purification apparatus according to another aspect of the second embodiment of the present disclosure. FIG. 7 is an elevated perspective view of the air purification apparatus according to another aspect of the second embodiment of the present disclosure. In the air purification apparatus 200 shown in FIG. 6, the main body 210 may be of cylindrical shape. That is, the main body 210 has an annular side face 213 and two opposing first and second ends 211, 212 coupled to the side face 213. To match the shape of the main body 210, the reactor sets 240 respectively have a disc-shaped casing 241 and the photocatalytic reactors 242 are filled in the disc-shaped casing 241. The light sources 230 may be disposed and distributed in a disc-shaped frame 232 sandwiched between the casings 241 of the reactor sets 240. The function of the air purification apparatus 200 shown in FIG. 7 is the same as that of the air purification apparatus 200 shown in FIG. 5, which will not be repeatedly described in detail herein.

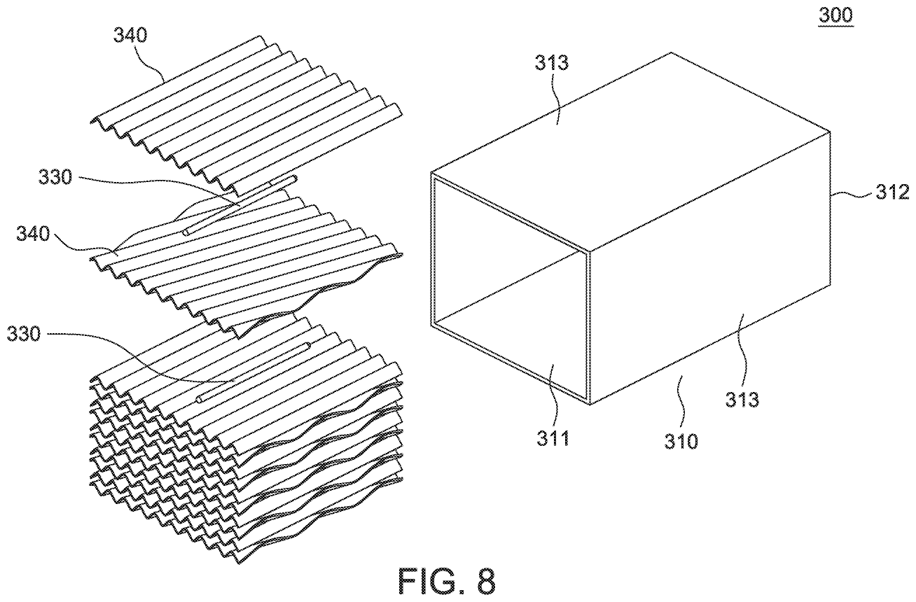

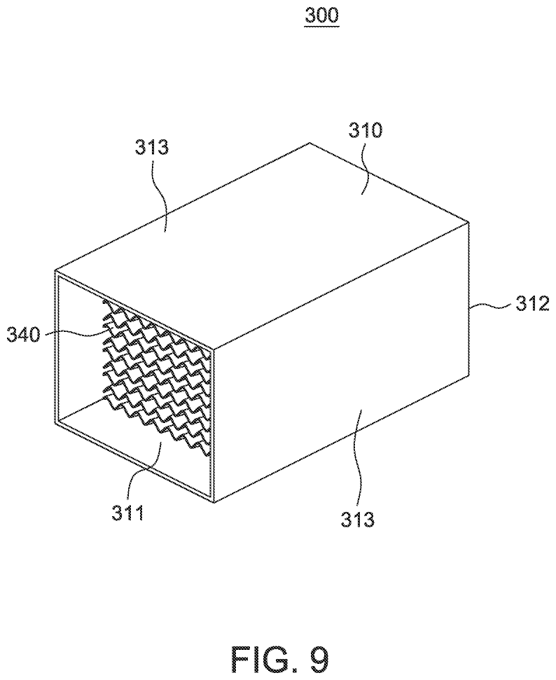

FIG. 8 is an exploded view of an air purification apparatus according to a third embodiment of the present disclosure. FIG. 9 is an elevated perspective view of the air purification apparatus according to the third embodiment of the present disclosure. The air purification apparatus 300 includes a main body 310, a plurality of light sources 330 and a plurality of photocatalytic reactors 340.

The main body 310 is hollow and of cuboidal shape. The main body 310 has a plurality of side faces 313 and two opposing first and second ends 311, 312 coupled to the side faces 313. The side faces 313 respectively have an inner surface that is a reflective surface for reflecting ultraviolet light. Further, the side faces 313 are arranged to stop air flow through. The first end 311 is an open end and configured to function as an air inlet. The second end 312 is an open end and configured to function as an air outlet. This means that air may enter the main body 310 from the first end 311 and be confined in the main body 310 by the side faces 313. The air finally leaves the main body 310 from the second end 312.

The photocatalytic reactors 340 are spaced from each other and disposed in the main body 310. The photocatalytic reactors 340 may respectively include a corrugated plate with a photocatalytic layer formed thereon. The corrugated plate may be arranged such that its extending direction is not parallel to the extending directions of adjacent upper and lower corrugated plates, as shown in the figure. The angle between two adjacent corrugated plates may be 10-45 degrees but the disclosure is not limited thereto. The photocatalytic layers may be formed on the photocatalytic reactors 340 by coating or spraying process. The photocatalytic layers on the photocatalytic reactors 340 may activate to reduce the foreign substances in the air through light energy.

The light sources 330 are respectively disposed between the photocatalytic reactors 340. In one embodiment, the light sources 330 may be ultraviolet lamps. The light sources 330 irradiate the photocatalytic reactors 340 so that the photocatalytic layers on the photocatalytic reactors 340 are activated by ultraviolet light to decontaminate or clean the air flowing pass the photocatalytic reactors 340.

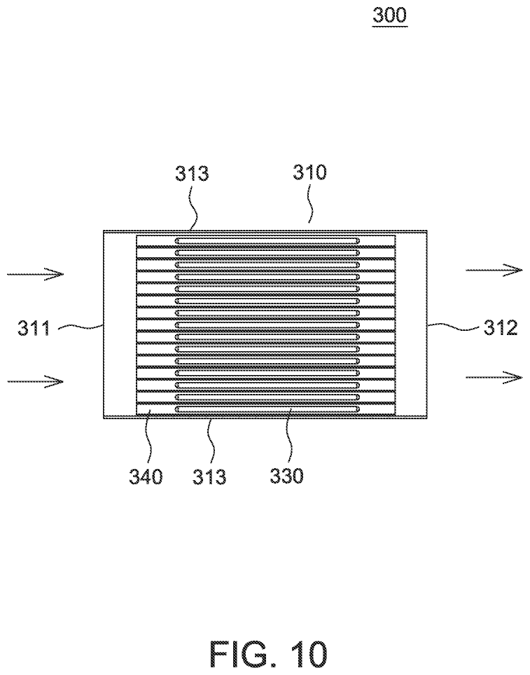

Now please refer to FIG. 10. In the air purification apparatus 300 of the present disclosure, air enters the air purification apparatus 300 from the first end 311 of the main body 310. The air may be inhaled into the main body 310 by a fan equipped in the main body 310 (not shown in the figure). The direction of arrows shown in the figure indicates that of the air flow. The air inhaled may pass through the space between the photocatalytic reactors 340. The light sources 330 irradiate the photocatalytic reactors 340 to activate the photocatalytic layers thereon. The inhaled air may contact and react with the activated photocatalytic layers on the photocatalytic reactors 340. Accordingly, the air inhaled is processed to be purified by the photocatalytic reactors 340. The purified air will finally leave the air purification apparatus 300 from the second end 312 of the main body 310.

In the air purification apparatus 300 of the present disclosure, the light sources 330 may be hot cathode tube lamps or cold cathode tube lamps. In other embodiments, the light sources 330 may be light-emitting diodes (LEDs). In other words, the light sources 330 may be selected from the group consisting of hot cathode tube lamps, cold cathode tube lamps, LEDs and combinations thereof. The light sources 330 may be ultraviolet lamps to generate ultraviolet light with a wavelength smaller than 450 nm to better activate the photocatalytic layers on the photocatalytic reactors 340.

In the air purification apparatus 300 of the present disclosure, the inner surfaces of the side faces 313 of the main body 310 may be reflective surfaces to reflect the ultraviolet light. This may utilize the ultraviolet light emitting from the light sources 330 more efficiently.

In the air purification apparatus 300 of the present disclosure, the bodies of the photocatalytic reactors 340 may be made of silicon dioxide or metal oxide through which light may penetrate or reflect. The metal oxide making up the bodies of the photocatalytic reactors 340 may be aluminum oxide, zirconium oxide, magnesium oxide or calcium oxide and combinations thereof. The photocatalytic layers on the photocatalytic reactors 340 may be made of titanium dioxide, zinc oxide or tungsten oxide and combinations thereof.

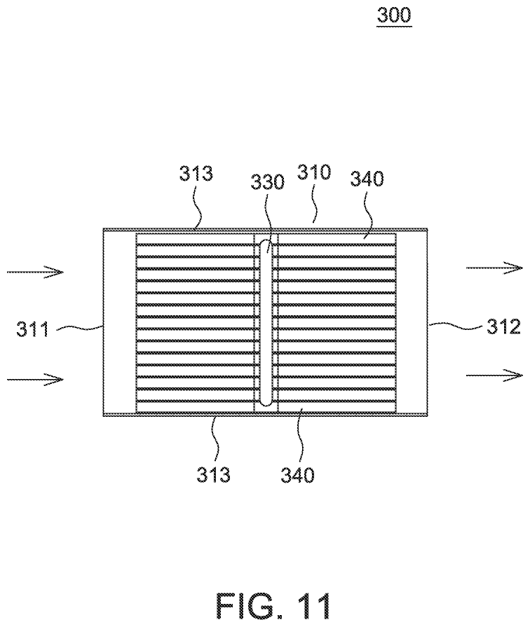

FIG. 11 is a schematic view of the air purification apparatus 300 according to another aspect of the third embodiment of the present disclosure. In the air purification apparatus 300 shown in FIG. 11, at least one light source 330 is inserted through the photocatalytic reactors 340 in the main body 310. The photocatalytic reactors 340 respectively have a hole formed therein and the at least one light source 330 is disposed through these holes. The function of the air purification apparatus 300 shown in FIG. 11 is the same as that of the air purification apparatus 300 shown in FIG. 10, which will not be repeatedly described in detail herein.

Please refer to Tables 1 and 2 below, which illustrate the effects of the air purification apparatus of the present disclosure in removing strain and contaminant. As shown in the Tables 1 and 2, the air purification apparatus of the present disclosure has good effects of removing strain and contaminant.

TABLE 1

| | | | final | |
|---|---|---|---|---|
| | | initial | concentration | |
| | strain or | concen- | (after 1~2 | removal |
| Item | contaminant | tration | hours) | rate (%) |
| 1 | covid-19 | 5.46 × 10⁵ | <1.0 × 10³ | 99.97 |
| 2 | human | 4.77 × 10⁶ | <1.6 × 10³ | 99.99 |
| | coronavirus | | | |
| 3 | H1N1 | 2.9 × 10⁸ | 2.96 × 10² | 99.99 |
| 4 | enterovirus 71 | 2.9 × 10⁸ | 3.24 × 10² | 99.99 |
| 5 | staphylococcus | 2.9 × 10⁸ | 200 | 97.9 |
| | aureus | | | |
| 6 | pseudomonas | 1.6 × 10⁸ | 1.0 × 10³ | 88.6 |
| | aeruginosa | | | |
| 7 | klebsiella | 1.6 × 10⁸ | 2.0 × 10³ | 76.6 |
| | pneumoniae | | | |
| 8 | formaldehyde | 10 ppmv | 3.5 ppmv | 65 |
| 9 | TVOCs | 10 ppmv | 5.44 ppmv | 45.6 |
| 10 | Ammonia | 10 ppmv | 5 ppmv | 50 |

TABLE 2

| | | concentration | | | | |
|---|---|---|---|---|---|---|
| | | | after | after | after | after |
| Item | strain or contaminant | initial | 1 hour | 2 hours | 4 hours | 8 hours |
| 1 | covid-19 | 100% | 0.30% | 0.00% | 0.00% | 0.00% |
| 2 | human coronavirus | 100% | 1.00% | 0.01% | 0.00% | 0.00% |
| 3 | H1N1 | 100% | 1.00% | 0.01% | 0.00% | 0.00% |
| 4 | enterovirus 71 | 100% | 1.00% | 0.01% | 0.00% | 0.00% |
| 5 | staphylococcus aureus | 100% | 2.10% | 0.04% | 0.00% | 0.00% |
| 6 | pseudomonas | 100% | 11.40% | 1.30% | 0.02% | 0.00% |
| | aeruginosa | | | | | |
| 7 | klebsiella pneumoniae | 100% | 23.40% | 5.48% | 0.30% | 0.00% |
| 8 | Formaldehyde | 100% | 35.00% | 12.25% | 1.50% | 0.02% |
| 9 | TVOCs | 100% | 54.40% | 29.59% | 8.76% | 0.77% |
| 10 | Ammonia | 100% | 50.00% | 25.00% | 6.25% | 0.39% |

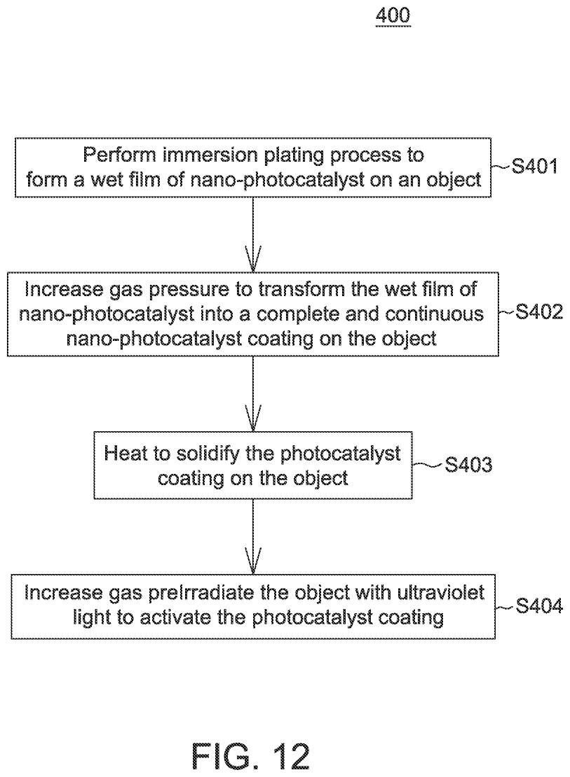

Referring to FIG. 12, which is a flow chart of a method 400 of manufacturing a photocatalytic reactor according to some embodiments of the present disclosure. The method 400 includes steps S401-S404. In one embodiment, the steps S401-S404 in the method 400 are not limited to the order listed in the flow chart. Some steps may be performed simultaneously, performed in an order other than that listed in the flow chart, or simply omitted. Additional steps may be performed as required. In one embodiment, the method 400 may be used to manufacture the photocatalytic reactors 140, 242 or 340 equipped in the air purification apparatus of the present disclosure.

Now please refer to FIG. 12. In step S401, an object to be coated with photocatalyst to form a photocatalytic reactor is rinsed or modified to be suitable for coating nano-photocatalytic sol-gel materials. Afterward, immersion plating is performed on the object so as to form a wet film of nano-photocatalytic sol with a thickness of not more than 200 μm on the object.

In step S402, the object formed with the wet film of nano-photocatalytic sol thereon is then disposed in a gas pressurized chamber. The gas pressure in the chamber is increased to more than 1 Kgf/cm² so that the nano-photocatalytic sol may be filled into all parts of the surface of the object. A complete and continuous nano-photocatalytic coating is thus formed on the surface of the object. After coating, the object is pre-dried at a temperature below 100° C.

In step S403, the object formed with the nano-photocatalytic coating is then heated to a temperature above 450° C. and maintained for more than 30 minutes to solidify the nano-photocatalytic coating.

In step S404, the object formed with the solidified nano-photocatalytic coating is then irradiated with ultraviolet light with a wavelength less than 300 nm and an energy greater than 1mW/cm² to activate the photocatalytic coating. The irradiation time should not be less than 300 seconds. A photocatalytic reactor is formed accordingly.

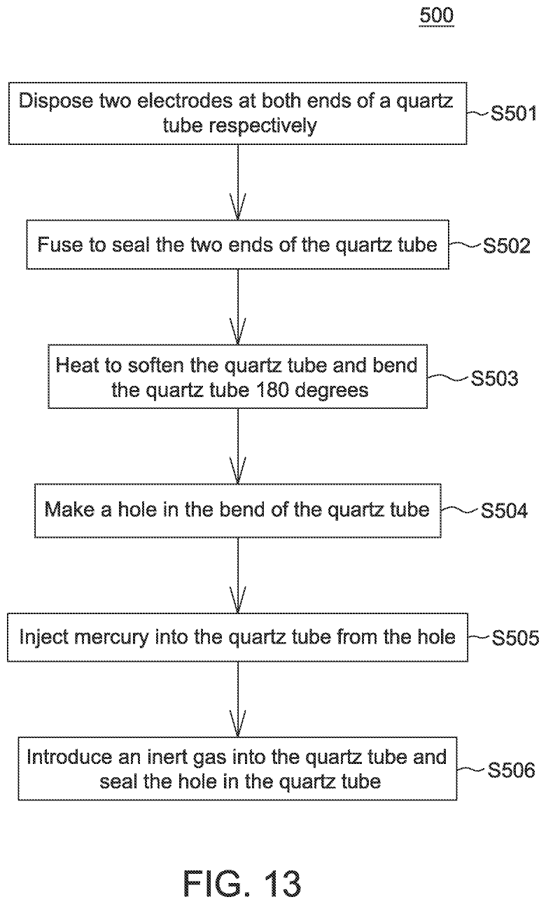

Referring to FIG. 13, which is a flow chart of a method 500 of manufacturing a mini cold cathode tube according to some embodiments of the present disclosure. The method 500 includes steps S501-S506. In one embodiment, the steps S501-S506 in the method 500 are not limited to the order listed in the flow chart. Some steps may be performed simultaneously, performed in an order other than that listed in the flow chart, or simply omitted. Additional steps may be performed as required. In one embodiment, the method 500 may be used to manufacture the light sources 130, 230 and 330 equipped in the air purification apparatus of the present disclosure when the light sources 130, 230 and 330 are cold cathode tubes.

Now please refer to FIG. 13. In step S501, two electrodes are disposed at both ends of a quartz tube, respectively. In one embodiment, the quartz tube may be one with a length less than 150 mm and an outer diameter less than 8 mm.

In step S502, the two ends of the quartz tube are fused to seal such that the electrodes are exposed out of the quartz tube.

In step S503, the quartz tube is heated to soften at the middle thereof and is then bent 180 degrees such that the two ends of the quartz tube is brought into being close to each other.

In step S504, a hole is made in the bend of the quartz tube. A syringe is then used to squeeze out less than 1 mg of mercury therefrom so that a mercury bead is form at a tip of a needle of the syringe.

In step S505, the mercury bead is injected into the quartz tube from the hole. More specifically, the needle of the syringe is inserted inclined into the hole in the bend of the quartz tube to have the mercury bead attached to an inner wall of the quartz tube. After the mercury bead is attached the inner wall of the quartz tube, the needle is then moved out of the quartz tube.

In step S506, the quartz tube with the mercury is disposed in a vacuum chamber. The vacuum chamber is then evacuated to a pressure of less than 1 Pa. Afterward, an inert gas is introduced to the evacuated vacuum chamber. The quartz tube is then fused to seal the hole in the bend of the quartz tube. A mini cold cathode containing trace mercury is formed accordingly.

In the method 500 of the present disclosure, the materials making up the quartz tube may be tuned so that the mini cold cathode manufactured may generate the ultraviolet light of different wavelength. For example, the mini cold cathode with a quartz tube made of pure quartz may generate the ultraviolet light with three wavelengths including 185 nm, 254 nm and 313 nm. The ultraviolet light with a wavelength of 185 nm may recombine oxygen molecules to form ozone to increase the sterilization effect. Alternatively, the quartz tube may be doped such that the mini cold cathode manufactured may generate the ultraviolet light with only two wavelengths including 254 nm and 313 nm.

The terms “first”, “second”, “third”, etc., used in this specification do not imply an order between elements or steps. In short, modifiers such as “first”, “second”, “third”, etc., in this specification and the appended claims are only used as reference words for different elements or steps, and are not intended to limit any function or to limit the chronological order.

Although the present disclosure has been disclosed by way of above embodiments, the embodiments are not intended to limit the present disclosure, and those skilled in the art will appreciate that changes and modifications may be made therein as long as those changes and modifications do not deviate from the spirit and the scope of the present disclosure. Therefore, the scope of the present disclosure should be construed according to the definitions in the appended claims.

What is claimed is:

1. An air purification apparatus, comprising:
an inner housing, the inner housing being porous to allow air flow to pass;
a plurality of photocatalytic reactors filled in the inner housing; the photocatalytic reactors respectively having a photocatalytic layer formed thereon; and
a light source disposed in the inner housing and surrounded by the photocatalytic reactors, the light source being configured to irradiate photocatalytic reactors to activate the photocatalytic layers on the photocatalytic reactors.

2. The air purification apparatus as claimed in claim 1, further comprising:
a main body receiving the inner housing, the main body having opposing first and second open ends configured to function as an air inlet and an air outlet respectively.

3. The air purification apparatus as claimed in claim 2, wherein the main body further has an inner surface configured to reflect light emitting from the light source.

4. The air purification apparatus as claimed in claim 2, wherein the main body further has a first annular side face coupled to the first and second open ends, the inner housing having a second annular side face facing the first annular side face, the second annular side face being porous.

5. The air purification apparatus as claimed in claim 1, wherein the light source is selected from the group consisting of a hot cathode tube lamp, a cold cathode tube lamp, a light bar composed of a plurality of light-emitting diodes (LEDs) and combinations thereof.

6. The air purification apparatus as claimed in claim 1, wherein the photocatalytic reactors have a shape of being selected from the group consisting of cube, ball, rod, fiber and sheet.

7. The air purification apparatus as claimed in claim 1, wherein the photocatalytic reactors comprise more than three layers of mesh structure, the mesh structure having an aperture size of less than 10 mm and misalignment of apertures in the layers of mesh structure being greater than 0.1 mm.

8. An air purification apparatus, comprising:
two reactor sets, the two reactor sets respectively having a casing and a plurality of photocatalytic reactors filled in the casing, the photocatalytic reactors respectively having a photocatalytic layer formed thereon, the casings being porous to allow air flow to pass; and
a plurality of light sources sandwiched between the two casings, the light sources being configured to irradiate photocatalytic reactors to activate the photocatalytic layers on the photocatalytic reactors.

9. The air purification apparatus as claimed in claim 8, further comprising:
a main body receiving the two reactor sets, the main body having opposing first and second open ends configured to function as an air inlet and an air outlet respectively.

10. The air purification apparatus as claimed in claim 9, wherein the main body further has an inner surface configured to reflect light emitting from the light sources.

11. The air purification apparatus as claimed in claim 8, further comprising:
a frame, the light sources being attached to the frame in a checkerboard shape.

12. The air purification apparatus as claimed in claim 11, wherein the light sources have more than two different wavebands that are alternately arranged in the checkerboard shape.

13. The air purification apparatus as claimed in claim 9, wherein the two casing are arranged to respectively face the air inlet and the air outlet.

14. The air purification apparatus as claimed in claim 8, wherein the photocatalytic reactors have a shape of being selected from the group consisting of cube, ball, rod, fiber and sheet.

15. The air purification apparatus as claimed in claim 8, wherein the photocatalytic reactors comprise more than three layers of mesh structure, the mesh structure having an aperture size of less than 10 mm and misalignment of apertures in the layers of mesh structure being greater than 0.1 mm.

16. An air purification apparatus, comprising:
a plurality of photocatalytic reactors spaced from each other, the photocatalytic reactors respectively comprising a corrugated plate with a photocatalytic layer formed thereon; and
at least one light source, the at least one light source being configured to irradiate photocatalytic reactors to activate the photocatalytic layers on the photocatalytic reactors.

17. The air purification apparatus as claimed in claim 16, further comprising:
a main body receiving the photocatalytic reactors, the main body having opposing first and second open ends configured to function as an air inlet and an air outlet respectively.

18. The air purification apparatus as claimed in claim 17, wherein the main body further has an inner surface configured to reflect light emitting from the at least one light source.

19. The air purification apparatus as claimed in claim 16, wherein the at least one light source includes a plurality of light sources respectively disposed between the photocatalytic reactors.

20. The air purification apparatus as claimed in claim 16, wherein the at least one light source is inserted through the photocatalytic reactors.
